(19) United States
(12) Patent Application Publication
(10) Pub. No.: US 2007/0185379 A1
(43) Pub. Date: Aug. 9, 2007

(54) Modular remote inspection device with digital imager

(75) Inventors: Tye Newman, Howell, MI (US); Al Boehnlein, Ypsilanti, MI (US); Paul J. Eckhoff, Fenton, MO (US); Alfred A. Pease, Ann Arbor, MI (US)

Correspondence Address: HARNESS, DICKEY & PIERCE, P.L.C., P.O. BOX 828, BLOOMFIELD HILLS, MI, 48303, US

(73) Assignee: Perceptron, Inc.

(21) Appl. No.: 11/645,280

(22) Filed: Dec. 22, 2006

Related U.S. Application Data
(63) Continuation-in-part of application No. 11/480,329, filed on Jun. 30, 2006.
Application No. 11/480,329 is a continuation-in-part of application No. 11/328,603, filed on Jan. 10, 2006.
Application No. 11/328,603 is a continuation-in-part of application No. 11/032,275, filed on Jan. 10, 2005.

(51) Int. Cl. A61B 1/04

(52) U.S. Cl. 600/110; 600/179; 600/109; 600/131; 600/139

(57) ABSTRACT

A remote inspection device includes a digital imager housing having a digital imaging device in communication with a digital video signal conversion device serializing the digital video signal. A digital display housing has a digital display in communication with a digital video signal re-conversion device de-serializing the digital video signal. A push stick housing is configured to be grasped by a user. A flexible cable interconnects the digital imager housing with the push stick housing, thereby rendering a position of the digital imager housing responsive to user manipulation of the push stick housing. The flexible cable also serves as a transmission medium transmitting the serialized digital video signal at least from the digital video signal conversion device to the push stick housing.

### CROSS-REFERENCE TO RELATED APPLICATIONS

This application is a continuation-in-part of U.S. patent application Ser. No. 11/480,329 filed on Jun. 30, 2006, which is in turn a continuation-in-part of U.S. patent application Ser. No. 11/328,603 filed on Jan. 10, 2006, which is in turn a continuation-in-part of U.S. patent application Ser. No. 11/032,275 filed on Jan. 10, 2005. The disclosures of the above applications are incorporated herein by reference in their entirety for any purpose.

### FIELD

The present disclosure relates generally to borescopes and video scopes.

### BACKGROUND

Borescopes and video scopes for inspecting visually obscured locations are typically tailored for particular applications. For instance, some borescopes have been tailored for use by plumbers to inspect pipes and drains. Likewise, other types of borescopes have been tailored for use by mechanics to inspect interior compartments of machinery being repaired.

The statements in this section merely provide background information related to the present disclosure and may not constitute prior art.

### SUMMARY

A remote inspection device includes a digital imager housing having a digital imaging device in communication with a digital video signal conversion device serializing the digital video signal. A digital display housing has a digital display in communication with a digital video signal re-conversion device de-serializing the digital video signal. A push stick housing is configured to be grasped by a user. A flexible cable interconnects the digital imager housing with the push stick housing, thereby rendering a position of the digital imager housing responsive to user manipulation of the push stick housing. The flexible cable also serves as a transmission medium transmitting the serialized digital video signal at least from the digital video signal conversion device to the push stick housing.

Further areas of applicability will become apparent from the description provided herein. It should be understood that the description and specific examples are intended for purposes of illustration only and are not intended to limit the scope of the present disclosure.

### DRAWINGS

FIG. 1 is a view of a modular remote inspection device with a digital imager and a digital display housing.

FIG. 2, including FIGS. 2A-C, is a set of block diagrams illustrating alternative functional components of the imager housing and the digital display housing of the modular remote inspection device.

FIG. 3 is a view of a modular remote inspection device with a remote digital display housing.

FIG. 4 is a cross-sectional view of an imaging device with light sources and a heat sink for use with a modular remote inspection device.

FIG. 5, including FIG. 5A and 5B, is a set of top and bottom views of a light source circuit board having apertures for passing thermal energy from light sources of an imaging device to a heat sink member of the imaging device.

The drawings described herein are for illustration purposes only and are not intended to limit the scope of the present disclosure in any way.

### DETAILED DESCRIPTION

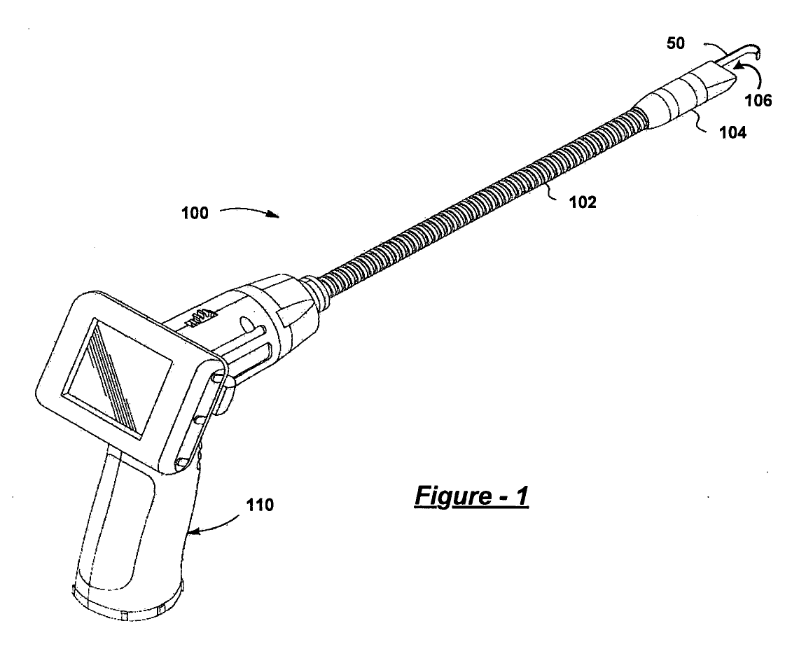

FIG. 1 illustrates an exemplary embodiment of a remote inspection device 100. The remote inspection device 100 is generally comprised of three primary components: a digital display housing 110, a digital imager housing 104, and a flexible cable 102 interconnecting the digital display housing 110 and the digital imager housing 104. The flexible cable 102 may be bent or curved as it is pushed into visually obscured areas, such as pipes, walls, etc. In an exemplary embodiment, the flexible cable 102 is a ribbed cylindrical conduit having an outer diameter in the range of 1 cm. The conduit can be made of either a metal, plastic or composite material. Smaller or larger diameters may be suitable depending on the application. Likewise, other suitable constructions for the flexible cable 102 are also contemplated by this disclosure.

The digital imager housing 104 is coupled to a distal end of the flexible cable 102. In the exemplary embodiment, the digital imager housing 104 is a substantially cylindrical shape that is concentrically aligned with the flexible cable 102. However, it is envisioned that the digital imager housing 104 may take other shapes. In any case, an outer diameter of the cylindrical digital imager housing 104 is preferably sized to be substantially equal to or less than the outer diameter of the flexible cable 102.

A digital imaging device 106 is embedded in an outwardly facing end of the cylindrical digital imager housing 104. The digital imaging device 106 captures an image of a viewing area proximate to the distal end of the flexible cable 102 and converts the image into a digital video signal. As defined herein, the digital imaging device 106 can be a purely digital imager, or it can be an analog imager having an analog to digital converter (ADC). In some embodiments, an attachment 50 can be removably coupled to the digital imager housing 14.

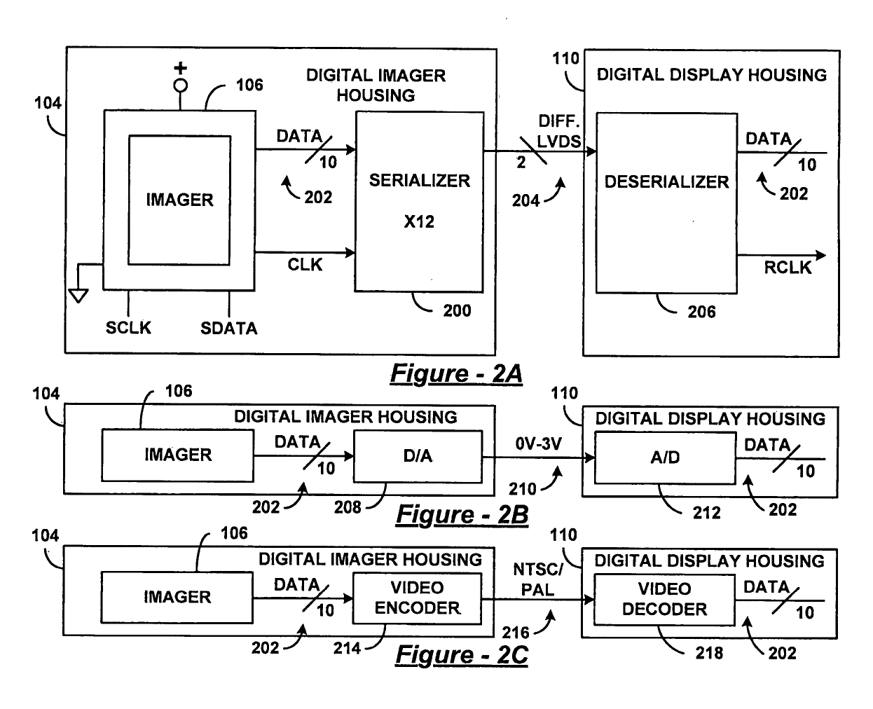

The digital imaging device 106 requires relatively more signal wires than a non-digital imaging device. Therefore, and referring now to FIG. 2, a digital video signal conversion device is included in order to serialize the digital video signal and thereby reduce the number of wire. In some embodiments, the conversion device is included in the digital imager housing 104 in order to reduce the number of wires required to be threaded through a portion of the flexible cable 102 (see FIG. 1). Alternatively, the conversion device can be located outside of the digital imager housing, but proximate to the digital imager 106 as opposed to the digital display. Therefore, it should be readily understood that an ADC and conversion device can be disposed in a push stick housing that is remote from a digital display housing in order to reduce a number of wires from the push stick housing to the digital display housing. In yet other embodiments, there is no need for a conversion device, especially if an ADC is in the display housing, or if the ADC is in the pushstick housing and the connection to a remote display housing is a wireless, digital connection. Therefore, it should be understood that the conversion device is used in some embodiments in order to reduce the number of wires needed to transmit digital video image data to the digital video display.

With particular reference now to FIG. 2A, the number of wires required to transmit the video signal from the digital imager housing to the digital display can be reduced from eighteen wires to eight wires by using a differential LVDS serializer 200 in the digital imager housing 104 to reformat the digital video signal 202 to a differential LVDS signal 204. Then, a differential LVDS deserializer 206 in the digital display housing 110 can receive the LVDS signal 204 and convert it back to the digital video signal 202 for use by the digital video display. In this case, the LVDS signal 204 replaces the twelve wires required to transmit the digital video signal with two wires required to transmit the LVDS signal. Six more wires are also required: one for power, one for ground, two for the LED light sources, one for a serial clock signal, and one for a serial data signal. One skilled in the art will recognize that the serial clock signal and the serial data signal are used to initiate the digital imaging device 106 at startup. In some additional or alternative embodiments, it is possible to reduce the number of wires even further by using a microcontroller to eliminate the serial communication lines, thereby reducing the wire count by an additional two wires.

Alternatively, and with particular reference to FIG. 2B, a digital to analog converter 208 in the digital imager housing 104 can convert the digital video signal 202 to an analog video signal 210. This analog video signal 210 can in turn be received by analog to digital converter 212 in the display housing 110, and be converted back to the digital video signal 202. Like use of a serializer, the use of the analog to digital converter reduces the number of wires from eighteen wires to eight wires. Again, two wires are needed to provide the analog voltage signal.

As another alternative, and with particular reference to FIG. 2C, the digital video signal 202 can be converted to an NTSC/PAL signal 216 by a video encoder 214 in the digital imager housing 108. One skilled in the art will readily recognize that NTSC is the standard for television broadcast in the United States and Japan, while PAL is its equivalent European standard. This NTSC/PAL signal 216 can then be reconverted to digital video signal 202 by video decoder 218 of display housing 110.

Returning the digital video signal to its original form allows use of a digital display to render the video captured by the digital imaging device 104. Use of the digital display can leverage various capabilities of such displays. For example, digital pan and zoom capability can be acquired by use of a larger imager in terms of pixels than the display, or by digital zoom. Thus, the display can be moved for greater detail/flexibility within the fixed visual cone of the imager head. Also, a software toggle can be implemented to increase perceived clarity and contrast in low spaces by switching from color to black and white.

Referring generally now to FIGS. 2A-C, it should be readily understood that the same types of conversion devices can be placed outside of the digital imager housing but proximate to the imager as opposed to the display. For example, each of the serializer 200, digital to analog converter 208, or video encoder 214 can be placed in a push stick housing that is remote from the digital imager housing and the digital display housing. This placement can be especially beneficial in the case of placement of an ADC in the push stick housing, and use of a wired connection between the push stick housing and the digital display housing.

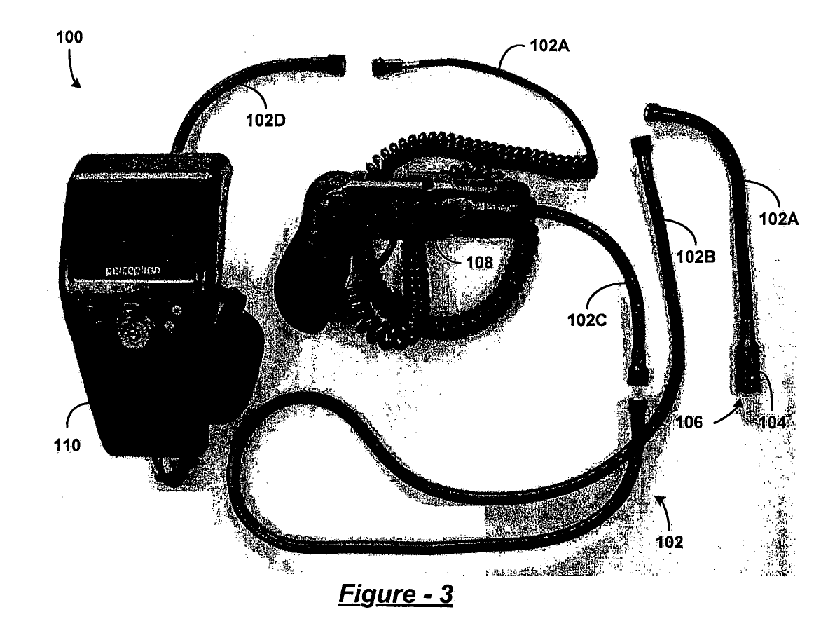

Turning now to FIG. 3, additional or alternative embodiments of the modular remote inspection device 100 can have a remote digital imager housing 110. In this instance, the remote housing 110 is configured to be held in another hand of the user of the inspection device 100, placed aside, or detachably attached to the user's person or a convenient structure in the user's environment. The flexible cable 102 can be attached to and/or passed through a push stick housing 108 that is configured to be grasped by the user. A series of ribbed cylindrical conduit sections 102A-C can connect the push stick housing 108 to the cylindrical digital imager housing 104. One or more extension sections 102B can be detachably attached between sections 102A and 102C to lengthen the portion of flexible cable 102 interconnecting push stick housing 108 and digital imager housing 104. It should be readily understood that the sections 102A-C can also be used in embodiments like those illustrated in FIG. 1 in which the digital display housing 110 is not remote, but is instead combined with push stick housing 108.

In some embodiments, as mentioned above, the flexible cable can pass through push stick housing 108 to digital display housing 110. For example, a coiled cable section 102D extending from push stick housing 108 can connect to a ribbed cylindrical conduit section 102E extending from digital display housing 110. Thus, flexible cable 102 can carry a serialized digital video signal from digital imaging device 106 through the ribbed cylindrical conduit sections 102A and 102C to push stick housing 108, through which it is transparently passed through to the remote digital video display housing 110 by the coiled cable section 102D and the ribbed cylindrical conduit section 102E. It should be readily understood that one or more extension sections 102B can be used to lengthen either or both of the cable portions interconnecting the push stick housing with the digital display housing and the digital imager housing.

In yet alternative or additional embodiments, flexible cable 102 can terminate at the push stick housing 108, and push stick housing can include a wireless transmitter device, thereby serving as a transmitter housing. In such embodiments, it should be readily understood that digital display housing 110 can contain a wireless receiver device, and the serialized digital video signal can be transmitted wirelessly from the push stick housing 108 to the digital display housing 110. It should also be readily understood that one or more antennas can be provided to the push stick housing 110 and the digital display housing to facilitate the wireless communication. Types of wireless communication can include Bluetooth, 802.11(b), 802.11(g), 802.11(n), wireless USB, Xigbee, analog, wireless NTSC/PAL, and others.

Two or more light sources protrude from the outwardly facing end of the cylindrical imager housing 104 along a perimeter of the imaging device 106 such that the imaging device 106 is recessed directly or indirectly between the light sources. In a presently preferred embodiment, the light sources are superbright LEDs, such as Nichias branded LEDs, which produce approximately twelve times the optical intensity compared to standard LEDs. Specifically, superbright LEDs such as 5mm Nichias LEDs produce upwards of 1.5 lumens each. The inclusion of the superbright LEDs produces a dramatic difference in light output, but also produces much more heat than standard LEDs. Therefore, an addition of a heat sink to the imager housing can be used to accommodate the superbright LEDs.

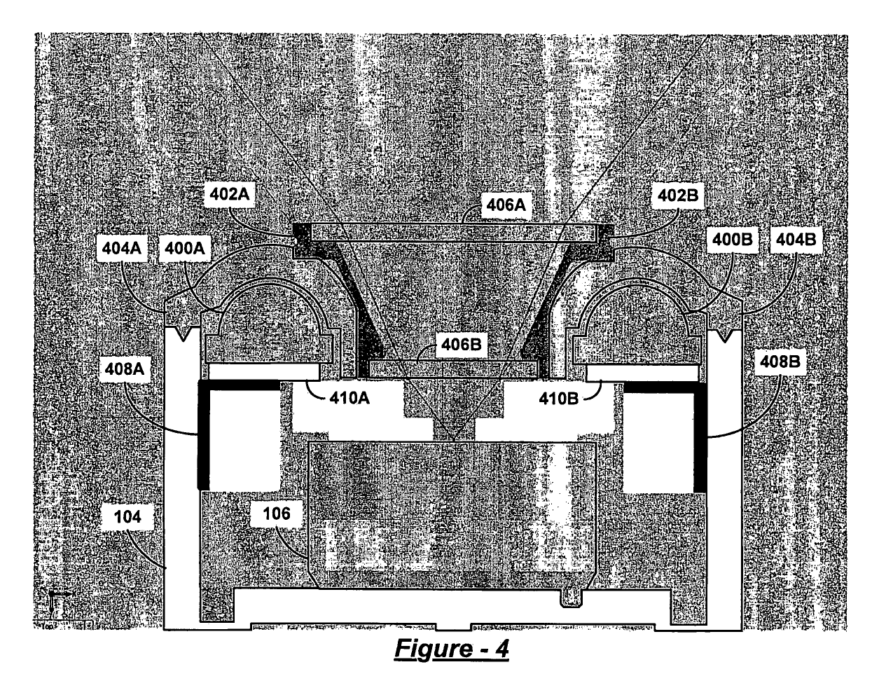

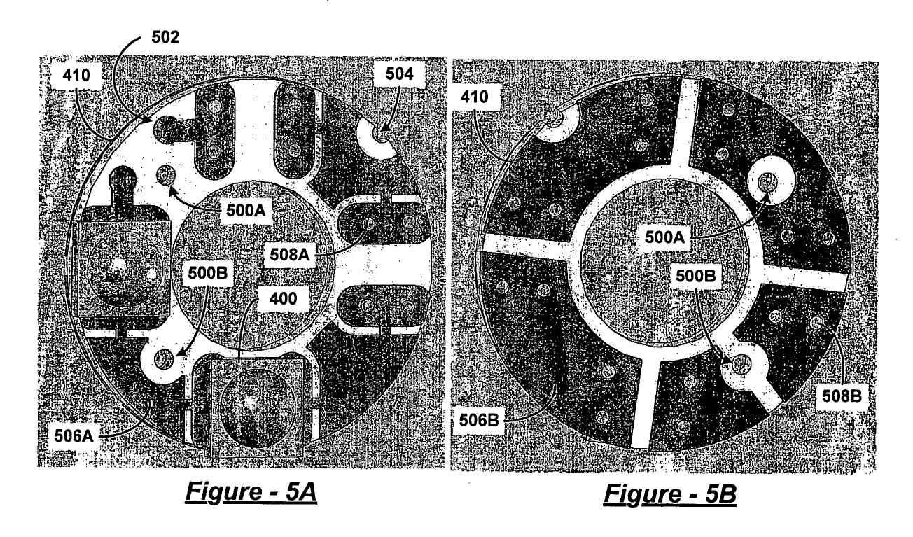

A transparent cap encases the imaging device and light sources within the imager housing. The transparent cap can also be modified to provide imaging optics (e.g., layered transparent imager cap) in order to effectively pull the focal point of the imaging device 106 outward compared to its previous location. For a given shape imager head, this change in the focal point can widen the effective field of view, thus rendering a snake formed of the flexible cable 102 and imager housing 104 more useful. This change in focal point can also allow vertical offset of the imaging device 106 from the light producing LEDs, thus making a smaller diameter imager head assembly possible. Additional details regarding the light sources, heat sink, and optics of the imager head are described below with reference to FIGS. 4 and 5.

It is envisioned that various types of imager housings 104 can be provided, each having different types of light sources and/or imaging optics that are targeted to different types of uses, or lack of light sources and imaging optics. For example, an imager housing 104 with light sources producing relatively greater amounts light in the infrared spectrum than another imager housing can be provided. For example, LEDs can be employed that produce light in the infrared spectrum, and one or more optical filters can be added to the imaging optics that selectively pass infra red light. This infrared imaging head is especially well suited to night vision and increasing the view distance and detail in galvanized pipe. In similar embodiments, the infrared light sources can be omitted to accomplish a thermal imaging head that has an infrared filter.

In additional or alternative embodiments, an imager housing 104 can be provided that has light sources optimized for producing light in the ultraviolet spectrum. For example, LEDs can be employed that produce light in the ultraviolet spectrum, with an optical filter provided to the imaging optics that selectively passes ultraviolet light. This ultraviolet imaging head is especially well suited for killing bacteria and fluorescing biological materials.

It should be readily understood that an imaging head can be provided that has white light sources, and that any or all of the different types of imaging heads can be supplied separately or in any combination. It is additionally envisioned that software for operating the digital display can have various modes for use with different types imager heads, and/or can have image processing capability to enhance images.

Turning now to FIG. 4, the digital imaging device 106 can be combined in imager housing 106 with light sources 400A-B. Light shield 402A-B prevents stray light from light sources 400A-B from entering the field of view of the digital imaging device 106. This light shield 402A-B can be attached to cap members 404A-B that protect the light sources 400A-B. Light shield 402A-B can also serve as a holder for holding one or more layers of imaging optics 406A-B, such as lenses or prisms, that shift the focus of the digital imaging device 106. Together, light the light shield 402A-B and imaging optics 406A-B permit placement of the imaging device beneath the light sources 400A-B, which allows for a slimmer imager housing 106. In some embodiments, the cap members 404A-B (e.g., an LED cover), the light shield 402A-B, and the imaging optics 406A-B can be positioned to ensure that an image 85° FOV is bent by a prism to be clear of the light shield 402A-B and the LED cover.

The imager head with light sources 400A-B that are superbright, such as superbright LEDs, can be provided with a metal housing 104 and heat sink member 408A-B. Heat sink member 408 can also be metal, and can permit transfer of heat produced by the light sources 400A-B to the metal housing 106 for dissipation. Heat sink member 408 can be shaped in an angular fashion (e.g., L-shaped) to facilitate passage of wires in the imager housing, and can have apertures to permit passage of wires to light source circuit board 41 OA-B.

Turning finally to FIGS. 5A and 5B, light source circuit board 410 can have apertures 500A-B, such as through holes, to permit passage of wires for powering light sources 400. These wires can be attached to pads 502. An index feature 504 can assist in ensuring proper orientation of the circuit board 410 within the metal housing. Pours 506A-B can be formed on the circuit board 410 to spread heat from the light sources 400 over a surface of the circuit board 410, and these pours can be made of any electrically non-conductive, but thermally conductive material, such as ceramic PCB. Vias 508A-B can transfer heat from a light source side of the circuit board 410 to a heat sink member side of the circuit board 410. Thus, the vias 508A-B thermally connect pours 506A-B on opposite surfaces of the circuit board 410 in order to transfer thermal energy produced by the light sources to the heat sink member.

The preceding description is merely exemplary in nature and is not intended to limit the present disclosure, application, or uses.

What is claimed is:

1. A remote inspection device, comprising:
a digital imager housing having a digital imaging device in communication with a digital video signal conversion device serializing the digital video signal;
a digital display housing having a digital display in communication with a digital video signal re-conversion device de-serializing the digital video signal;
a push stick housing configured to be grasped by a user; and
a flexible cable interconnecting the digital imager housing with the push stick housing, thereby rendering a position of the digital imager housing responsive to user manipulation of the push stick housing, wherein the flexible cable also serves as a transmission medium transmitting the serialized digital video signal at least from the digital video signal conversion device to the push stick housing.

2. The remote inspection device of claim 1, wherein the digital display housing is the push stick housing, and the flexible cable is directly connected to the digital video signal re-conversion device.

3. The remote inspection device of claim 1, wherein the digital display housing is remote from the push stick housing, and the flexible cable further extends from the push stick to the digital display housing, where it is directly connected to the digital video signal re-conversion device.

4. The remote inspection device of claim 1, wherein the digital display housing is remote from the push stick housing, and the flexible cable is connected to a wireless transmitter disposed in the push stick housing that wirelessly transmits the serialized digital video signal to a wireless receiver that is disposed in the digital display housing and connected to the digital video signal re-conversion device.

5. The remote inspection device of claim 1, wherein the digital video signal conversion device is a differential LVDS serializer converting the digital video signal to a differential LVDS signal, and the digital video signal re-conversion device is a differential LVDS de-serializer converting the differential LVDS signal back to the digital video signal.

6. The remote inspection device of claim 1, wherein the digital video signal conversion device is an analog to digital converter converting the digital video signal to an analog video signal, and the digital video signal re-conversion device is an analog to digital converter converting the analog video signal back to the digital video signal.

7. The remote inspection device of claim 1, wherein the digital video signal conversion device is a video encoder converting the digital video signal to a television broadcast signal conforming to a television broadcast format, and the digital video signal re-conversion device is a video decoder converting the television broadcast signal back to the digital video signal.

8. The remote inspection device of claim 1, wherein: (1) the imager housing is cylindrical in shape and composed primarily of metal; (2) two or more light sources are disposed to emit light from an end of the digital imager housing; (3) the imaging device is disposed within the imager housing and oriented toward the light sources to: (a) receive light passing between the light sources; (b) capture an image of a viewing area proximate to the end of the imager housing; and (c) convert the image into a video signal; and (4) a heat sink member is disposed to collect thermal energy produced by the light sources and transmit the thermal energy to the metal of the imager housing for dissipation.

9. The remote inspection device of claim 8, further comprising a plurality of imager heads each having a respective imager housing and imaging device, wherein the imaging device captures an image of a viewing area proximate to a distal end of the imager housing and converts the image into a video signal, each of the imager heads is detachably attachable to the flexible cable, and at least two of the imager heads are not identical at least in terms of presence or types of light sources disposed therein.

10. The remote inspection device of claim 1, further comprising a plurality of imager heads each having a respective imager housing and imaging device, wherein the imaging device captures an image of a viewing area proximate to a distal end of the imager housing and converts the image into a video signal, each of the imager heads is detachably attachable to the flexible cable, and at least two of the imager heads are not identical at least in terms of presence or types of light sources disposed therein.

11. An imager head assembly for use with a remote inspection device, the imager head assembly comprising:
a cylindrical imager housing composed primarily of metal;
two or more light sources disposed to emit light from an end of the cylindrical imager housing;
an imaging device disposed within the imager housing and oriented toward the light sources, thereby receiving light passing between the two or more light sources, capturing an image of a viewing area proximate to the end of the imager housing, and converting the image into a video signal; and
a heat sink member disposed to collect thermal energy produced by the light sources and transmit the thermal energy to the metal of the imager housing for dissipation.

12. The imager head of claim 11, wherein the light sources are superbright LEDs each emitting the light at an intensity of at least 1.5 lumens.

13. The imager head of claim 11, wherein a circuit board for powering the light sources has vias permitting transfer of thermal energy produced by the light sources to the heat sink member.

14. The imager head of claim 11, wherein a circuit board for powering the light sources has at least one thermally conductive pours on one or more surfaces of the circuit board in order to spread heat from the light sources over the one or more surfaces of the circuit board.

15. The imager head of claim 11, further comprising a light shield that prevents light from the light sources from directly impinging imaging optics of the imaging device, thereby preventing a bright spot in the image captured by the imaging device.

16. The imager head of claim 15, wherein said imaging optics adjust a focal point of the imaging device.

17. The imager head of claim 11, further comprising imaging optics adjusting a focal point of the imaging device to position the imaging device beneath the light sources, as opposed to directly between the light sources, thereby permitting a separation distance of the light sources to be less than a width of the imaging device and achieving a slimmer imaging head.

18. The imager head of claim 11, wherein the imaging device is a digital imaging device and is in communication with a digital video signal conversion device serializing the digital video signal.

19. The imager head of claim 1 1, wherein the light sources emit infrared light, and imaging optics of the imaging head include a filter selectively passing infrared light to the imaging device.

20. The imager head of claim 11, wherein the light sources emit ultraviolet light, and imaging optics of the imaging head include a filter selectively passing ultraviolet light to the imaging device.

21. A remote inspection device, comprising:
a plurality of imager heads each having a cylindrical imager housing and an imaging device disposed within the imager housing, wherein the imaging device captures an image of a viewing area proximate to a distal end of the imager housing and converting the image into a video signal;
a display housing having a video display receiving a video signal from the imaging device;
a push stick housing configured to be grasped by a user; and
a flexible cable interconnecting the imager housing with the push stick housing, thereby rendering a position of the imager housing responsive to user manipulation of the push stick housing, wherein the flexible cable also serves as a transmission medium transmitting the video signal from the imaging device,
wherein each of the imager heads is detachably attachable to the flexible cable, and at least two of the imager heads are not identical in terms of presence or type of light sources disposed therein.

22. The remote inspection device of claim 21, wherein one of the imager heads has light sources and the other does not have light sources, the imager head that does not have light sources having an optical filter that selectively passes infrared light to its respective imaging device.

23. The remote inspection device of claim 21, wherein one of the imaging heads has light sources that all emit visible light, and another of the imaging heads has light sources that all selectively emit either infrared light or ultraviolet light.

24. The remote inspection device of claim 23, wherein the other imaging head has infrared light sources and an infrared filter selectively passing infrared light to its respective imaging device.

25. The remote inspection device of claim 23, wherein the other imaging head has ultraviolet light sources and an ultraviolet filter selectively passing ultraviolet light to its respective imaging device.

26. The remote inspection device of claim 21, wherein one of the imaging heads has an infrared filter selectively passing infrared light to its respective imaging device, and light sources that all selectively emit infrared light, and another of the imaging heads has an ultraviolet filter selectively passing ultraviolet light to its respective imaging device, and light sources that all selectively emit ultraviolet light.

27. The remote inspection device of claim 21, wherein the display housing has image processing software and a software toggle for switching between image processing modes, the image processing modes including at least two of:
(a) an infrared image processing mode for processing images captured by an imager head having light sources that emit infrared light;
(b) a thermal image processing mode for processing images captured by an imager head having no light sources;
(c) an ultraviolet light image processing mode for processing images captured by an imager head having light sources that emit ultraviolet light; or
(d) a visible light image processing mode for processing images captured by an imager head having light sources that emit visible light.

28. The remote inspection device of claim 21, wherein each imaging device of the heads is a digital imaging device and is in communication with a digital video signal conversion device serializing the digital video signal within its respective head, and the video display is a digital display in communication with a digital video signal re-conversion device de-serializing the digital video signal within the display housing.

29. The remote inspection device of claim 21, wherein, for at least one of the heads: (1) the imager housing is cylindrical in shape and composed primarily of metal; (2) two or more light sources are disposed to emit light from an end of the digital imager housing; (3) the imaging device is disposed within the imager housing and oriented toward the light sources to: (a) receive light passing between the light sources; (b) capture an image of a viewing area proximate to the end of the imager housing; and (c) convert the image into a video signal; and (4) a heat sink member is disposed to collect thermal energy produced by the light sources and transmit the thermal energy to the metal of the imager housing for dissipation.

30. A remote inspection device, comprising:
a plurality of imager heads each having a respective imager housing and digital imaging device disposed therein in communication with a digital video signal conversion device serializing the digital video signal, wherein the imaging device captures an image of a viewing area proximate to a distal end of the imager housing and converts the image into a video signal;
a push stick housing configured to be grasped by a user;
a digital display housing remote from the push stick housing and having a digital display in communication with a digital video signal re-conversion device de-serializing the digital video signal; and
a flexible cable interconnecting the digital imager housing with the push stick housing, thereby rendering a position of the digital imager housing responsive to user manipulation of the push stick housing, wherein the flexible cable also serves as a transmission medium transmitting the serialized digital video signal at least from the digital video signal conversion device to the push stick housing,
wherein each of the imager heads is detachably attachable to the flexible cable, and at least two of the imager heads are not identical at least in terms of presence or types of light sources disposed therein, and
wherein: (1) at least one digital imager housing of the heads is cylindrical in shape and composed primarily of metal; (2) one or more light sources are disposed to emit light from a distal end of that digital imager housing; (3) its respective imaging device is disposed within that digital imager housing and oriented toward the light sources to: (a) receive light passing between the light sources; (b) capture an image of a viewing area proximate to the distal end of the imager housing; (c) and convert the image into a video signal; and (4) a heat sink member is disposed to collect thermal energy produced by the light sources and transmit the thermal energy to the metal of that imager housing for dissipation.

31. The remote inspection device of claim 30, wherein the flexible cable is connected to a wireless transmitter disposed in the push stick housing that wirelessly transmits the serialized digital video signal to a wireless receiver that is disposed in the digital display housing and connected to the digital video signal re-conversion device.

32. The remote inspection device of claim 31, wherein the wireless transmitter and wireless receiver employ at least one wireless transmission protocol selected from the following:
(1) Bluetooth;
(2) 802.11(b);
(3) 802.11(g);
(4) 802.11(n);
(5) wireless USB;
(6) Xigbee;
(7) analog; or
(8) wireless NTSC/PAL.

33. The remote inspection device of claim 30, wherein the flexible cable further extends from the push stick housing to the digital display housing and is connected directly to the digital video signal re-conversion device.

34. The remote inspection device of claim 30, wherein the digital display has pan and zoom capability.

35. The remote inspection device of claim 30, wherein the digital display has a software toggle for switching between a color display mode and a black and white display mode.
